(19) United States
(12) Patent Application Publication
(10) Pub. No.: US 2021/0077574 A1
(43) Pub. Date: Mar. 18, 2021

(54) PHYCOCYANIN COMPOSITION FOR USE IN INHIBITING BONE RESORPTION

(71) Applicant: INSTITUT NATIONAL DE LA RECHERCHE AGRONOMIQUE (INRA), Paris (FR)

(72) Inventors: Véronique COXAM, Ceyrat (FR); Marie-Jeanne DAVICCO, Chamalieres (FR); Laurent LEOTOING, Romagnat (FR); Fabien WAUQUIER, Tauves (FR)

(73) Assignee: INSTITUT NATIONAL DE LA RECHERCHE AGRONOMIQUE (INRA), Paris (FR)

(21) Appl. No.: 16/620,287

(22) PCT Filed: Jun. 5, 2018
(86) PCT No.: PCT/EP2018/064714

§ 371 Date: Dec. 6, 2019

(30) Foreign Application Priority Data
Jun. 6, 2017 (FR) 1755014

(51) Int. Cl. A61K 38/16; A23L 33/135; A61K 35/748; A61P 19/10; A23L 33/18

(52) U.S. Cl. CPC A61K 38/164; A23L 33/135; A61K 9/0053; A61P 19/10; A23L 33/18; A61K 35/748

(57) ABSTRACT

The present invention relates to a phycocyanin composition for use in inhibiting bone resorption in humans or animals.

### FIELD OF THE INVENTION

The present invention relates to the field of the inhibition of bone resorption in humans or animals.

### PRIOR ART

Bone is not a static tissue. Bone undergoes constant remodeling by destruction and de novo synthesis of bone tissue in a complex process involving two main types of cells, namely osteoblasts, which produce new bone tissue, and osteoclasts, which break down bone.

Throughout life, bone tissue remodeling coordinates bone resorption and bone formation and thus ensures renewal of the skeleton, safeguarding its structure. When the system is in equilibrium, for example before the menopause, it ensures the integrity of the mechanical strength of the skeleton by preventing excessive aging of its components. This essential function is provided by the cellular interaction of the osteoclasts and osteoblasts. The remodeling process always begins with resorption, followed after a certain delay by bone formation.

The osteoblasts, the cells responsible for bone formation, differentiate from precursor cells and express and secrete structural proteins such as type 1 collagen, as well as enzymes (alkaline phosphatase) and numerous regulatory peptides and BMPs (Bone Morphogenetic Proteins) (Stein G. et al., 1990, Curr. Opin Cell Biol. Vol. 2: 1018-1027; Harris S et al., 1994, J. Bone Miner Res Vol. 9: 855-863).

The osteoclasts are multinucleate cells that are responsible for bone loss, in a process commonly called bone resorption.

In the growth period of humans or animals, the activity of bone formation predominates: it is the acquisition of the bone pool.

At adult age, in humans or animals, the balanced action of the osteoblasts and osteoclasts allows the bone mass to be maintained over time and at the same time ensures bone tissue remodeling by bone resorption and de novo synthesis.

However, with age, an imbalance develops in the bone remodeling process, leading to bone loss, which is called osteopenia.

Age-related osteopenia is a universal phenomenon, not pathological per se, but which constitutes the field of osteoporosis since reduction of bone mass is the essential etiological factor in the genesis of this disorder, which does not, however, rule out the influence of other parameters such as, for example, the architecture of the skeleton or the tendency to fall.

In fact, if exacerbated, osteopenia leads to risk of fracture (mineral density below which the slightest shock is likely to cause a fracture), which determines the development of osteoporosis.

Therefore osteoporosis (postmenopausal or senile) may be conceived of as a childhood pathology, prophylaxis of which could be based both on (i) optimization of the bone pool (acquired during the individual's growth), and on (ii) slowing of age-related bone loss.

A considerable heterogeneity is observed in the value of the peak bone mass from one individual to another, owing to variations in the process of bone growth from a very young age. Thus, the maximum bone mass reached before the age of 30 years, also called peak bone mass, is very variable from one individual to another. As they age, individuals with a low value of peak bone mass are at a disadvantage.

Consequently, early management of the individual must be favored, since the two critical phases for the bone pool are:
the period of growth, allowing acquisition of the maximum bone mass (peak bone mass);
aging, determining the rate of loss of bone mass.

Among the pathological disorders linked to an imbalance of bone metabolism, we may notably mention osteoporosis, Paget's disease, bone loss, or osteolysis observed near a prosthesis.

There are other factors that may increase bone loss and lead to osteoporosis, such as cigarette smoking, alcohol abuse, a sedentary lifestyle, a low calcium intake, an unbalanced diet, or a deficiency of vitamin D.

These different causes of increased bone loss must be distinguished from the causes connected with disorders such as cancer or diabetes.

Among the disorders associated with abnormal bone resorption, the commonest is osteoporosis, the most frequent manifestation of which is observed in women, after the onset of the menopause.

Osteoporosis is a systemic skeletal disease characterized by a decrease in bone mass and deterioration of the microarchitecture of the bone tissue, associated with an increase in brittleness of the bone and its susceptibility to fractures.

According to the clinical classification adopted by the World Health Organization (WHO), the state of bone health of an individual is determined by the value of the bone mineral density (BMD), as measured by osteodensitometry, compared against a predetermined normal value, this predetermined normal value being that of the mean value of the peak of bone mineral density of the population aged 30 years.

Two types of osteoporosis are distinguished, namely type I and type II.

Type I osteoporosis is six times more frequent in women that in men. Type I osteoporosis occurs in particular in a subgroup of postmenopausal women, aged from 51 to 75 years, and is characterized by exaggerated bone loss, predominant in the trabecular bone. Fractures of the vertebral bodies and of the lower end of the forearm are the usual complications.

Type I osteoporosis is mainly associated with estrogenic hormone deficiency of the menopause, and to a certain extent also with the andropause.

Type II osteoporosis affects a wide population of men and women over 70 years and is associated with fractures of the neck of the femur, of the upper end of the humerus and of the tibia, i.e. bone regions containing both cortical bone and trabecular bone. The circulating levels of parathormone (PTH) are often high.

Type II osteoporosis is two times more frequent in women.

Strategies aiming to restore estrogen impregnation (and androgen impregnation in men) by hormone replacement therapy were initially strongly encouraged, notably to attenuate the cohort of functional signs of the menopause, and especially to reduce the vascular and osseous risks caused by the hormone deficiency.

Nevertheless, for various reasons (contraindications, reluctance to prescribe hormones, increased risks of cancer, etc.), this type of prophylactic treatment is no longer the first-line treatment. Healthcare professionals are therefore currently relatively powerless in terms of prophylactic tools. Treatments connected with insufficiency of bone remodeling are particularly required among those that inhibit bone resorption. A variety of agents that inhibit bone resorption are known, and are prescribed notably in the treatment of osteoporosis, notably of age-related osteoporosis.

Among bone resorption inhibitors, selective estrogenic receptor modulators (SERMs) are known, such as raloxifene. However, this compound is ineffective for fractures of the upper end of the femur (More Study) and it is contraindicated in patients with a history of thromboembolism. Furthermore, this treatment increases the frequency of hot flashes. Synthetic steroids are also known, such as tibolone, which possesses estrogen and progestogen activity, with a weak androgenic property, but which may cause leukorrhea, vaginitis, mastodynia, as well as weight gain.

In fact, there are now a great many compounds that act on inhibition of bone resorption, among which we may mention the bisphosphonate family, such as etidronate or alendronate (European Patent No. EP 210 728, US patent application published under No. 2001/0046977), thioamide oxazolidinones (US patent application published under No. 2002/0010341), or the isoflavones (US patent application published under No. 2002/0035074).

Among the agents commonly used as bone resorption inhibitors, we may mention alendronate, risedronate, ibandronate and zoledronic acid. We may also mention denosumab, which is a monoclonal antibody targeting the RANK/RANKL system. The marketing authorizations of these various compounds as medicinal products are restricted to a therapeutic indication for osteoporosis. These bone resorption inhibitors are not currently prescribed for osteoporosis prevention. Moreover, the use of these bone resorption inhibitors has a variety of side-effects such as (i) undesirable gastrointestinal effects for alendronate, risedronate and ibandronate; (ii) buccal lesions and osteonecrosis of the jaw, or even fractures, for zoledronic acid, and (iii) cardiac disorders for denosumab. Although there is now a relatively wide range of compounds for stimulating bone formation and/or inhibiting bone resorption, healthcare professionals are constantly in need of new active compounds, notably because of the limited success of the current treatments and the important side-effects associated with them.

Moreover, taking into account the chronic character of certain conditions caused by an imbalance of bone metabolism, there is a need for new active compounds that can be used for a long period of time in humans or animals.

That is why health professionals, as well as the official regulatory bodies (report on osteoporosis in the European Community, 1998), recommend integrating validated complementary, or even alternative, therapies. A nutritional approach notably meets these criteria in full.

There is therefore an expressed need for new means intended for preventing or treating imbalance of bone metabolism, in particular new means for preventing or treating bone resorption, in particular age-related bone resorption in humans or animals.

### SUMMARY OF THE INVENTION

The present invention relates to a phycocyanin composition, for use in inhibiting bone resorption in humans or animals.

In certain embodiments, said composition is a nutritional composition suitable for oral administration. Advantageously, said nutritional composition aims to prevent bone loss, also called osteopenia, in particular in individuals in whom the occurrence of bone loss may be expected, for example on account of age or else bone loss that is likely to occur owing to other factors, for example such as taking medicinal products.

In certain other embodiments, said composition is a pharmaceutical composition for human or veterinary use.

In certain embodiments, said nutritional composition or said pharmaceutical composition is intended to prevent bone loss, also called osteopenia. In certain embodiments, said nutritional composition or said pharmaceutical composition is intended to prevent the bone loss that occurs with aging or on account of taking medicinal products, or to prevent bone loss occurring in certain diseases such as obesity, diabetes, thyroid disorders and disorders of the adrenal glands.

In certain embodiments, said nutritional composition is suitable for administration, in particular for oral administration, of a daily amount from 0.01 to 10 000 mg of the compound phycocyanin.

In certain embodiments, said nutritional composition is suitable for daily administration, in particular for daily oral administration, of from 0.05 mg/kg to 1000 mg/kg, advantageously from 1 mg/kg to 200 mg/kg, which includes from 10 mg/kg to 100 mg/kg.

Note that on average, it is considered that an adult man or woman weighs about 80 kg.

In certain embodiments, said pharmaceutical composition is suitable for administration, in particular for oral administration, of a daily amount from 1 mg to 10 000 mg of the compound phycocyanin, which includes adaptation to daily administration ranging from 4 mg to 8000 mg.

In certain embodiments, said pharmaceutical composition is suitable for daily administration, in particular for daily oral administration, ranging from 0.05 mg/kg to 1000 mg/kg, advantageously from 1 mg/kg to 200 mg/kg, and preferably from 10 mg/kg to 100 mg/kg.

A phycocyanin composition according to the invention may be intended to prevent or treat any type of osteopenia.

In certain embodiments, said composition is intended to prevent or treat a pathology selected from type I or type II osteoporosis, secondary osteoporoses, Paget's disease, bone loss or osteolysis observed near a prosthesis.

### DESCRIPTION OF THE FIGURES

FIG. 1 illustrates the results of measurement of bone mass in different groups of animals. On the ordinate: values of bone mineral density (BMD), expressed in grams per cubic centimeter (g/cm³ or g/cc). On the abscissa, the results for each of the groups of animals, from left to right in FIG. 1: (i) SH-Control: control group that underwent sham surgery; (ii) OVX-Control: control group that underwent an ovariectomy; (iii) OVX-Spirulina: ovariectomized group that received a diet supplemented with lysed spirulina; (iv) OVX-phycocyanin: ovariectomized group that received a diet supplemented with purified phycocyanin.

FIG. 2 illustrates the results of measurement of the proliferation of a cell line of mouse pre-osteoblasts (MC3T3-E1 line). On the ordinate: osteoblast proliferation index (corresponding to the cellular mitochondrial activity expressed as the change in optical density per hour (Δ OD/h)). On the abscissa, the pre-osteoblast culture conditions, from left to right in FIG. 2: (i) C10%: culture medium supplemented with 10% (v/v) of fetal bovine serum; (ii) C2%: culture medium supplemented with 2% (v/v) of fetal bovine serum; (iii) Ph100: culture medium supplemented with 100 μg/ml of purified phycocyanin, (iv) Ph250: culture medium supplemented with 250 μg/ml of purified phycocyanin, (v) Ph1000: culture medium supplemented with 1000 μg/ml of purified phycocyanin, (vi) Ph2500: culture medium supplemented with 2500 μg/ml of purified phycocyanin.

FIG. 3 illustrates the results of proliferation of a line of mouse osteoclasts (RAW264.7 line) exposed to different culture conditions. On the ordinate: osteoclast proliferation index (corresponding to the cellular mitochondrial activity expressed as change in optical density per minute (Δ DO/min)). On the abscissa, the osteoclast culture conditions, from left to right in FIG. 3: (i) C2%: culture medium supplemented with 2% (v/v) of fetal bovine serum; (ii) C10%: culture medium supplemented with 10% (v/v) of fetal bovine serum; (iii) Phy5: culture medium supplemented with 5 μg/ml of purified phycocyanin, (iv) Phy10: culture medium supplemented with 10 μg/ml of purified phycocyanin, (v) Phy25: culture medium supplemented with 25 μg/ml of purified phycocyanin, (vi) Phy100: culture medium supplemented with 100 μg/ml of purified phycocyanin, (vii) Phy250: culture medium supplemented with 250 μg/ml of purified phycocyanin, (vi) Phy1000: culture medium supplemented with 1000 μg/ml of purified phycocyanin.

FIG. 4 illustrates measurements of the enzymatic activity of tartrate-resistant acid phosphatase (TRAP) expressed as osteoclasts (cells of the Raw 264.7 line) in culture. On the ordinate: TRAP activity, expressed as the change in optical density per hour and per milligram of proteins (Δ OD/h/mg proteins). On the abscissa, from left to right in FIG. 5: the following osteoclast culture conditions: (i) C: Culture medium only; (ii) RL: culture medium supplemented with 50 ng/ml of RANK-L; (iii) RL5: RL medium supplemented with 5 μg/ml of purified phycocyanin; (iv) RL10: RL medium supplemented with 10 μg/ml of purified phycocyanin; (v) RL25: RL medium supplemented with 25 μg/ml of purified phycocyanin; (vi) RL50: RL medium supplemented with 50 μg/ml of purified phycocyanin; (vii) RL100: RL medium supplemented with 100 μg/ml of purified phycocyanin; (iii) C100: medium alone supplemented with 100 μg/ml of purified phycocyanin.

FIG. 5 illustrates measurements of the enzymatic activity of tartrate-resistant acid phosphatase (TRAP) expressed by osteoclasts (cells of the Raw 264.7 line) in culture. On the ordinate: TRAP activity, expressed as the change in optical density per hour and per milligram of proteins (Δ OD/h/mg proteins). On the abscissa, from left to right in FIG. 5: the following osteoclast culture conditions: (i) C: culture medium only; (ii) RL: culture medium supplemented with 50 ng/ml of RANK-L; (iii) group of two bars. Bar on the right (gray): RL5: RL medium supplemented with 5 μg/ml of purified phycocyanin; bar on the left (black): RL medium supplemented with the same dose of a 4-times purer phycocyanin (Sigma) (assay based on colorimetric analysis); (iv) group of two bars. Bar on the right (gray): RL10: RL medium supplemented with 10 μg/ml of purified phycocyanin; bar on the left (black): RL medium supplemented with the same dose of a 4-times purer phycocyanin (Sigma) (assay based on colorimetric analysis); (v) group of two bars. Bar on the right (gray): RL25: RL medium supplemented with 25 μg/ml of purified phycocyanin; bar on the left (black): RL medium supplemented with the same dose of a 4-times purer phycocyanin (Sigma) (assay based on colorimetric analysis) (vi) group of two bars. Bar on the right (gray): RL50: RL medium supplemented with 50 μg/ml of purified phycocyanin; bar on the left (black): RL medium supplemented with the same dose of a 4-times purer phycocyanin (Sigma) (assay based on colorimetric analysis).

FIG. 6 illustrates expression of the gene coding for TRAP in cultures of osteoclasts of the RAW 264.7 line. On the ordinate: expression level of the gene coding for TRAP, expressed as fold increase. On the abscissa, from left to right in FIG. 7, the different culture conditions to which the cells are exposed: (i) Control: culture medium only; (ii) RL: culture medium supplemented with 50 ng/ml of RANK-L; (iii) RL+Phy5: RL medium supplemented with 5 μg/ml of purified phycocyanin; (iv) RL+Phy10: RL medium supplemented with 10 μg/ml of purified phycocyanin; (v) RL+Phy25: RL medium supplemented with 25 μg/ml of purified phycocyanin; (vi) RL+Phy50: RL medium supplemented with 50 μg/ml of purified phycocyanin; (vii) RL+Phy100: RL medium supplemented with 100 μg/ml of purified phycocyanin.

FIG. 7 illustrates expression of the gene coding for Nox4 in cultures of osteoclasts of the RAW 264.7 line. On the ordinate: expression level of the gene coding for Nox4, expressed as fold increase. On the abscissa, from left to right in FIG. 8, the different culture conditions to which the cells are exposed: (i) Control: culture medium only; (ii) RL: culture medium supplemented with 50 ng/ml of RANK-L; (iii) RL+Phy5: RL medium supplemented with 5 μg/ml of purified phycocyanin; (iv) RL+Phy10: RL medium supplemented with 10 μg/ml of purified phycocyanin; (v) RL+Phy25: RL medium supplemented with 25 μg/ml of purified phycocyanin; (vi) RL+Phy50: RL medium supplemented with 50 μg/ml of purified phycocyanin; (vii) RL+Phy100: RL medium supplemented with 100 μg/ml of purified phycocyanin.

FIG. 8 illustrates expression of the gene coding for NFATC1 in cultures of osteoclasts of the RAW 264.7 line. On the ordinate: expression level of the gene coding for Nox4, expressed as fold increase. On the abscissa, from left to right in FIG. 9, the different culture conditions to which the cells are exposed: (i) Control: culture medium only; (ii) RL: culture medium supplemented with 50 ng/ml of RANK-L; (iii) RL+Phy5: RL medium supplemented with 5 μg/ml of purified phycocyanin; (iv) RL+Phy10: RL medium supplemented with 10 μg/ml of purified phycocyanin; (v) RL+Phy25: RL medium supplemented with 25 μg/ml of purified phycocyanin; (vi) RL+Phy50: RL medium supplemented with 50 μg/ml of purified phycocyanin; (vii) RL+Phy100: RL medium supplemented with 100 μg/ml of purified phycocyanin.

### DETAILED DESCRIPTION OF THE INVENTION

The inventors have shown that phycocyanin has an inhibitory effect on osteoclastic activity in in-vivo and in-vitro models of induced deregulation of bone metabolism.

Phycocyanin is a protein in the family of phycobiliproteins, which are present in certain cyanobacteria. Phycocyanin consists of two protein subunits, namely the alpha and beta subunits. Phycocyanin comprises a plurality of prosthetic chromophore groups of the bilin type, these chromophore groups being bound to phycocyanin covalently, via thioether bonds, to cysteine residues. Phycocyanin comprises a bilin group bound to the alpha subunit and two bilin groups bound to the beta subunit. Phycocyanin is present in cyanobacteria, such as Spirulina platensis, in the form of a complex mixture of trimers, hexamers and decamers (Romay et al., 2003, Current Protein and Peptide Science, Vol. 4: 207-216).

Phycocyanin is thus a protein found in extracts obtained from cyanobacteria of the genus Spirulina, and in particular from Spirulina platensis. Spirulina extracts have a complex composition. Such extracts classically comprise from 50 to 70 wt % of proteins, from 4 to 7 wt % of lipids (83% of saponifiable fraction and 17% of unsaponifiable fraction), from 15 to 25 wt % of carbohydrates, relative to the weight of dry matter. Spirulina extracts also comprise water-soluble vitamins (e.g. vitamins B1, B2, B6, B9, B12 and vitamin C), fat-soluble vitamins (e.g. beta-carotene, tocopherols), pigments, numerous minerals and trace elements (e.g. calcium, phosphorus, magnesium, iron, zinc, copper, chromium, manganese, sodium, potassium). The protein richness of spirulina extracts is utilized qualitatively in food supplements, owing to the presence of all of the essential amino acids (isoleucine, leucine, lysine, methionine, phenylalanine, tryptophan and valine). Spirulina extracts are known to have a variety of beneficial properties for health, including stimulation of the immune system, antiviral, anticancer, antioxidant, and detoxifying properties, as well as effects against hyperlipidemia. Spirulina extracts are currently marketed as food supplements, on account of their many claimed beneficial effects. However, spirulina extracts may also lead to undesirable effects, such as induction of a change in bone metabolism, leading to a reduction of bone mineral density, in particular in individuals having a hormone deficiency, and more specifically in individuals having an estrogen deficiency (Ishimi et al., Biosci Biotechnol Biochem, Vol. 70 (No. 2): 363-368).

Therefore the inventors showed, quite unexpectedly, that phycocyanin, which is one of the predominant protein components in spirulina extracts, possesses, in contrast to spirulina extracts, properties of inhibition of the reduction of bone metabolism that is observed in individuals with estrogen deficiency.

More precisely, the inventors have shown that a phycocyanin composition possesses properties of inhibition of osteoclastic activity in individuals with estrogen deficiency. Thus, it is shown in the examples that a phycocyanin composition makes it possible to inhibit the effect of reduction of bone mineral density (demineralization process) in individuals having an estrogen deficit. It is notably shown that individuals given a phycocyanin composition display an increase in the level of the protein marker OPG (osteoprotegerin), compared to individuals not given this composition. It is also shown that there is an improvement of the OPG/RANKL ratio, RANKL (for “Receptor Activator of Nuclear Factor Kappa B Ligand”) being a protein synthesized by the osteoblasts that acts on the differentiation and activation of the osteoclasts when it binds to the RANK receptor. The increase in the expression level of the OPG marker as well as in the OPG/RANK-L ratio in the individuals who received a phycocyanin composition is indicative of an inhibitory effect on the differentiation and activation of the osteoclasts and on a favorable orientation of bone metabolism (toward bone formation).

It has also been shown that a phycocyanin composition induces inhibition of proliferation and differentiation of osteoclasts in vitro. Notably, a phycocyanin composition inhibits a protein marker of osteoclastic activity such as tartrate-resistant acid phosphatase (TRAP).

It has also been shown that a phycocyanin composition inhibits the expression of genes that are markers of osteoclast differentiation, such as the genes trap (coding for the protein TRAP) and nfatc1 (coding for the protein NFATC1), as well as of genes that are indicative of oxidative stress, such as nox4 (coding for the protein NOX4).

The inventors have also shown that a phycocyanin composition induces proliferation of osteoblasts.

Moreover, as is shown in the examples, phycocyanin does not display a detectable effect imitating the activity of a compound of the estrogen type, also called “estrogen-like” effect, as evidenced by the low weight of the uterine horns, as well as the absence of weight gain of the female rats treated. These results confirm the benefit of phycocyanin for prevention or treatment of bone resorption in humans or animals, quite particularly in a hormone deficiency situation. On account of the absence of an “estogen-like” effect caused by a phycocyanin composition in the sense of the invention, such a composition may be administered indiscriminately (i) to men or to women and (ii) to any nonhuman animal, including any nonhuman mammal, of male or female gender.

These experimental results all show that administration of a phycocyanin composition has a beneficial action on bone metabolism in individuals with estrogen deficiency. These results show that a phycocyanin composition makes it possible to prevent, at least partly, bone loss that is caused by a reduction of estrogen impregnation.

Without wishing to be bound by any theory, the inventors think that the activity of a phycocyanin composition on the bone metabolism of individuals with estrogen deficiency is mainly due to an activity of inhibition of bone resorption, although part of the activity of a phycocyanin composition may also be due to an activity of stimulation of bone formation, since induction of osteoblast proliferation is also shown.

The present invention relates to a phycocyanin composition for use in inhibiting bone resorption in humans or animals.

In certain embodiments, said phycocyanin composition is a nutritional composition. In preferred embodiments of a nutritional phycocyanin composition, such a composition is suitable for oral administration.

“Phycocyanin” means, according to the invention, the protein referenced under CAS No. 11016-15-2. Preferably, it is a phycocyanin derived from a cyanobacterium of the species Spirulina platensis.

“Phycocyanin” means the whole protein, which does not include peptide fragments of this protein.

“Phycocyanin composition” means, according to the invention, a phycocyanin-enriched composition, i.e. a composition comprising at least 40 wt % of phycocyanin, relative to the total dry weight of the composition.

In the present description, “dry weight” means the weight of dry matter of said composition. The weight of dry matter may be determined by a person skilled in the art by any known technique, including after removal of the water by stove drying of the composition until there is complete evaporation of the water that was initially contained therein. The stove drying step may be carried out conventionally at a temperature varying from 103° C. to 110° C. at atmospheric pressure.

According to the present description, a phycocyanin composition comprising at least 40 wt % of phycocyanin, relative to the dry weight of the composition, includes the phycocyanin compositions comprising at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99 wt % of phycocyanin, relative to the total dry weight of the composition.

According to this definition, a phycocyanin composition comprises at most 60 wt % of compounds other than phycocyanin, relative to the dry weight of the composition, which includes the phycocyanin compositions comprising at most 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% and 1 wt % of compounds other than phycocyanin, relative to the total dry weight of the composition.

In a phycocyanin composition according to the invention, the compounds other than phycocyanin may be compounds present initially in the starting material that was used, for example in the starting material derived from spirulina, before applying a method of extraction and phycocyanin enrichment from the crude starting material.

A variety of compositions comprising at least 40 wt % of phycocyanin are available commercially. We may notably mention the following compositions: (i) C-Phycocyanin marketed by the company Sigma-Aldrich under reference No. P2172 and No. P6161, (ii) C-Phycocyanin (Spirulina sp) marketed by the company Prozyme (Hayward, Canada) under reference No. PB11, (iii) C-phycocyanin marketed by the company Soley Institute (Istanbul, Turkey) under reference A620/280.

Phycocyanin can also be obtained by extraction from certain cyanobacteria such as the cyanobacterium Arthrospira platensis, by methods that are known by a person skilled in the art. To obtain a starting phycocyanin composition from Arthrospira platensis, a person skilled in the art may notably refer to the methods described by Moraes et al. (2011, Brazilian Journal of Chemical Engineering, Vol. 28 (No. 1): 45-49), Kamble et al. (2013, J Applied Pharmaceutical Science, Vol. 3 (No. 8): 149-153), Sivasankari et al. (International Journal of Current Microbiology and Applied Sciences, Vol. 3 (No. 8): 904-909), Slimane et al. (2014, Bull Inst Natn Scien Tech Mer de Salammbo, Vol. 42: 13-15); Kumar et al. (2014, J Plant Physiol, Vol. 19 (No. 2): 184-188), Manirafasha et al. (2017, Journal of Applied Phycology,: 1-10, doi: 10.1007/s10811-016-0989-y), Paswan et al. (2016, J Fluoresc. Vol. 26(2): 577-83. doi: 10.1007/s10895-015-1742-7); Chethana et al. (2015, J Food Sci Technol. Vol. 52(4): 2415-21. doi: 10.1007/s13197-014-1287-9), Seo et al. (2013, Int J Mol Sci. Vol.14(1): 1778-87. doi: 10.3390/ijms14011778) or in PCT application No. WO 2014/045177.

Inhibition of bone resorption means, according to the invention, inhibition of the activity of destruction of bone tissue by osteoclasts. To verify that administration of phycocyanin, in humans or animals, inhibits bone resorption, a person skilled in the art may for example measure the urinary excretion of deoxypyridinoline, a decrease in excretion of deoxypyridinoline reflecting inhibition of bone resorption (in: The biological markers of bone remodeling: pre-analytical variations and recommendations for use thereof. P. Garnero, F. Bianchi, P. C. Carlier, V. Genty, N. Jacob, S. Kamel, C. Kindermans, E. Pluvier, M. Pressac & J. C. Souberbielle. Annales de Biologie Clinique, 58 (6), 683-704 (2000)). Preferably, for measuring inhibition of bone resorption, a person skilled in the art can measure the serum level of another protein marker, collagen type 1 cross-linked C-telopeptide (CTX): For measuring the serum level of CTX, a person skilled in the art may refer to the article by Ganero et al. (2017, Mol Diagn Ther. doi: 10.1007/s40291-017-0272-1). For measuring the serum level of CTX, a person skilled in the art may for example use commercial measurement kits, such as the CTX kit (Osteoporosis test) marketed by the company LifeLabs®, the CTX test kit marketed by the company Roche, the ELISA test kit marketed under reference No. LS-F20998 by the company LifeSpan BioSciences Inc., or the ELISA test kit marketed under reference No. AC-02F1 by the company Immunodiagnosticsystem (IDS).

A nutritional or therapeutic composition of phycocyanin as active compound is intended firstly for preventing bone loss due to an imbalance in bone tissue remodeling, in humans or animals, in particular in a nonhuman mammal, notably a domestic mammal such as a dog or a cat, equines such as horses, or in animals other than mammals such as avian species, which includes the farmed avian species such as hens, geese, ducks, turkeys, pigeons etc.

The phycocyanin composition is notably intended for individuals who have symptoms of bone deficiency (osteopenia), or are likely to suffer from bone deficiency, i.e. an imbalance of the ratio of bone formation to bone resorption, which, if it continues, induces a decrease in bone mass.

To determine whether a subject has a state of osteopenia (reduced bone mass), and consequently requires administration of phycocyanin, a person skilled in the art may notably refer to the report of the World Health Organization (WHO) from 1994 titled Assessment of fracture risk and its application to screening for postmenopausal osteoporosis WHO Technical Series-843.

A nutritional or pharmaceutical composition intended for human or veterinary use according to the invention is useful for preventing the bone loss that occurs with aging.

In particular, a nutritional or pharmaceutical composition intended for human or veterinary use according to the invention is useful in the treatment of physiological situations such as type I or type II osteoporosis, secondary osteoporoses, Paget's disease, bone loss or osteolysis observed near a prosthesis.

The secondary osteoporoses include bone resorption that is caused in physiological situations induced in diseases such as osteosarcoma, rheumatoid arthritis, rheumatoid spondylarthritis, malabsorption syndromes, hypogonadism, primary hyperparathyroidism, diabetes, obesity, chronic obstructive pulmonary disease, chronic liver disease, chronic kidney failure, neurological disorders, AIDS, untreated hyperthyroidism and osteomalacia. Thus, treatment of secondary osteoporoses relates to treatment of physiological situations of bone resorption which are not the cause of the disease to be treated but are a physiological imbalance concomitant to the disease to be treated. In other words, administration of a phycocyanin composition as defined in the present description to a patient with a specific pathology does not aim to exert a therapeutic effect on the disease itself. Administration of a phycocyanin composition to a patient therefore has the aim of inhibiting the bone resorption that is concomitant to the occurrence of said pathology.

A phycocyanin composition according to the invention may be in the form of a nutritional composition or else in the form of a pharmaceutical composition, as is described below.

### Nutritional Compositions of Phycocyanin

As already mentioned above, many disorders associated with an imbalance of bone metabolism, such as osteoporosis, develop progressively over a long period of time and require continuous treatment. They may therefore be prevented or treated by regular administration of phycocyanin, preferably in the form of a nutritional composition.

In particular, regular nutritional intake of phycocyanin is useful for preventing the bone loss that occurs in the course of aging.

The invention relates to a nutritional composition for inhibiting bone resorption, characterized in that it comprises phycocyanin, as an active nutritional compound.

“Nutritional composition” means a composition comprising, as active agent against bone resorption, a phycocyanin composition as defined in the present description and constituting a food composition or else a food supplement not possessing the characteristics of a medicinal product.

A nutritional composition according to the invention may comprise from 1 wt % to 90 wt % of a phycocyanin composition as defined in the present description.

The various uses of phycocyanin for making a nutritional composition will be defined below in relation to the technical characteristics of said nutritional composition.

A nutritional composition according to the invention is preferably suitable for oral administration.

According to a first aspect, a nutritional composition according to the invention is a dietetic food used for maintaining good health of humans or animals ingesting it. A nutritional composition of this kind is also commonly called functional food, which is intended to be consumed either as an integral part of the diet, or as a food supplement, but whose phycocyanin content implies a physiological role going beyond supplying the basic nutritional requirements. A nutritional composition according to the invention includes phycocyanin-enriched food compositions.

According to certain aspects, said nutritional composition is intended to prevent the bone loss that occurs with aging (osteopenia).

According to other aspects, said nutritional composition is intended for preventing or treating disorders associated with an imbalance of the ratio of bone formation to bone resorption.

According to yet other aspects, said nutritional composition is intended for preventing physiological disorders selected from osteopenia, type I or type II osteoporosis, secondary osteoporoses, Paget's disease and the bone loss or osteolysis observed near a prosthesis.

A nutritional composition that comprises a phycocyanin composition as defined in the present description may be in a great variety of forms of food compositions and drinks, including juices (of fruits or vegetables or algae), vegetable milks, oils, butters, margarines, vegetable fats, canned food (for example tuna in oil), soups, milk-based preparations (yoghurts, fromage frais), ice creams, cheeses (for example cheeses preserved in oil), baked goods (such as bread, biscuits, crepes and cakes), desserts, confectionery products, cereal bars, breakfast cereals, condiments, products for seasoning foodstuffs (notably spices and sauces).

In certain embodiments, a nutritional composition according to the invention may be in liquid form, for example in the form of an aqueous solution or in the form of an oily solution.

In other embodiments, a nutritional composition according to the invention may be in solid form, for example in the form of powder. Notably, the nutritional composition may be lyophilized and may be in the form of a powder.

A nutritional composition according to the invention may also be in the form of a great variety of products intended as animal feed, whether they are in wet form, in semi-wet form or in dry form, notably in the form of pellets or granules.

In certain embodiments of a nutritional composition according to the invention, the phycocyanin composition as defined in the present description and which is comprised in said nutritional composition is in the form of an extraction product obtained from a cyanobacterium, belonging to the Spirulina family, and in particular Spirulina platensis.

In other embodiments, a nutritional composition according to the invention is in the form of any product, in particular any drink, for example a flavored drink.

According to another aspect, in a nutritional composition according to the invention, the phycocyanin composition as defined in the present description may be produced by extraction or by synthesis. We may in particular use a recombinant phycocyanin produced by host cells transfected or transformed by a polynucleotide comprising an expression cassette coding for phycocyanin.

Preferably, a nutritional composition according to the invention comprises an amount of phycocyanin suitable for daily oral administration varying from 0.05 mg to 10 000 mg.

As an illustration, in embodiments in which a nutritional composition is produced using a phycocyanin composition having a phycocyanin content of 50 wt % relative to the total weight of the phycocyanin composition, a nutritional composition is obtained comprising 1000 mg of phycocyanin by including 2000 mg of phycocyanin composition in the nutritional composition being prepared.

For human consumption, a nutritional composition according to the invention comprises an amount of phycocyanin suitable for a daily intake of phycocyanin, supplied by said composition, varying from 1 mg to 10 000 mg, advantageously from 4 mg to 8000 mg, which includes from 5 mg to 6000 mg.

The results of the examples demonstrate efficacy of a phycocyanin composition as defined in the present description, for inhibiting bone resorption, when said composition is administered in mice in a daily amount of phycocyanin of 1 mg, or at a daily dose of 500 mg per kilogram of live weight.

A person skilled in the art is able to determine, using known methods, the effective doses in other mammals, including in humans (Hemon et al., Conference “Science et décision en santé environmentale” [Science and decision in environmental health], September 1996, Metz, France.ineris-00971997; Meyer C., ed. sc., 2017, Dictionnaire des Sciences Animales [Dictionary of Animal Sciences]. [On line]. Montpellier, France, Cirad. [21 May 2017]. <URL:http://dico-sciences-animales.cirad.fr/>).

According to these known methods, this signifies that a phycocyanin composition as defined in the present description is effective, for inhibiting bone resorption in humans, at a daily dose of phycocyanin of 55 mg per kilogram of live weight, or a daily dose of phycocyanin of (i) 4400 mg for a human weighing 80 kg and of (ii) 5500 mg for a human weighing 100 kg.

Thus, in certain embodiments, a phycocyanin composition as defined in the present description is suitable for daily administration in humans, of an amount of phycocyanin from 4 mg to 10 000 mg, notably depending on the weight of the individual in question.

By way of illustration, a phycocyanin composition as defined in the present description is effective, for inhibiting bone resorption in cats, at a daily dose of phycocyanin of 100 mg per kilogram of live weight.

As a further example, a phycocyanin composition as defined in the present description is effective, for inhibiting bone resorption in dogs, at a daily dose of phycocyanin of 70 mg per kilogram of live weight.

As another illustration, a phycocyanin composition as defined in the present description is effective, for inhibiting bone resorption in horses, at a daily dose of phycocyanin of 30 mg per kilogram of live weight.

In general, a phycocyanin composition as defined in the present description is preferably suitable for daily administration of phycocyanin from 0.05 mg/kg to 1000 mg/kg, which includes from 1 mg/kg to 200 mg/kg, for example from 10 mg/kg to 100 mg/kg.

According to yet another aspect, the aforementioned nutritional composition may comprise other nutritional compounds, in combination with phycocyanin.

In certain embodiments, in particular in the embodiments in which the phycocyanin composition is prepared by purification of phycocyanin starting from extracts, quite especially starting from extracts of cyanobacteria, the phycocyanin composition may comprise, besides the protein phycocyanin, also compounds that are likely to consist of nutritional compounds, such as vitamins, lipids, carbohydrates, trace elements, mineral salts, or other phyto-constituents, which were initially contained in a crude starting extract. In other embodiments, the nutritional composition comprises other nutritional compounds, which have been added to the starting phycocyanin composition.

Thus, the nutritional composition according to the invention may also comprise a source of calcium, for example in the form of a physiologically acceptable organic or inorganic compound, such as inorganic calcium salts (calcium chloride, calcium phosphate, calcium sulfate, calcium oxide, calcium hydroxide or calcium carbonate) or organic components containing calcium such as skim milk powder, calcium caseinate or else organic calcium salts (calcium citrate, calcium maleate or mixtures thereof).

The amount of calcium contained in a nutritional composition according to the invention is suitable for daily administration, supplied by said composition, of between 100 mg and 1000 mg, preferably between 200 mg and 700 mg and quite preferably between 300 mg and 600 mg of calcium.

A nutritional composition according to the invention may also comprise vitamins, such as vitamin A, vitamin D, vitamin E, vitamin K, vitamin C, folic acid, thiamine, riboflavin, vitamin B6, vitamin B12, niacin, biotin, or pantothenic acid.

A nutritional composition according to the invention may also comprise mineral elements and trace elements such as sodium, potassium, phosphorus, magnesium, copper, zinc, iron, selenium, chromium and molybdenum.

It may also comprise soluble fibers such as agar-agar, an alginate, carob, carrageenan, gum arabic, guar gum, karaya gum, pectin or xanthan gum, these soluble fibers being in hydrolyzed or nonhydrolyzed form.

It may further comprise proteins, for example proteins with nutritional value, such as hydrolyzates of proteins, in particular hydrolyzates of milk proteins, isolates of milk proteins containing micellar casein, hydrolyzates of meat proteins, vegetable proteins, etc.

It may also comprise compounds that are a source of energy, notably one or more sources of carbohydrates selected from maltodextrins, starch, lactose, glucose, sucrose, fructose, xylitol, fermentable sugars such as inulin and fructo-oligosaccharides, sorbitol and optionally fatty acids such as omega-3.

Furthermore, a nutritional composition according to the invention may also comprise spices, aromatic herbs or else other micronutrients such as other polyphenols, sterols, phenolic acids, stilbenes, carotenoids, iridoids, organosulfur compounds and terpenes.

As already mentioned above, a phycocyanin composition intended for inhibiting bone resorption according to the invention may also be comprised in a pharmaceutical composition, as described hereunder.

### Human or Veterinary Pharmaceutical Compositions According to the Invention

The invention also relates to a human or veterinary pharmaceutical composition for use in inhibiting bone resorption, characterized in that it comprises, as active principle, a phycocyanin composition as defined in the present description.

In particular, the invention relates to the use of a phycocyanin composition for making a pharmaceutical composition for human or veterinary use for preventing or treating a pathology associated with an imbalance of bone metabolism, and quite especially a pharmaceutical composition able to inhibit bone resorption.

The uses of a phycocyanin composition for making a pharmaceutical composition will be described in relation to the technical characteristics of said pharmaceutical composition hereunder.

A pharmaceutical composition according to the invention comprises, as active principle, a phycocyanin composition as defined in the present description, in an amount suitable for inhibiting bone resorption in individuals requiring such a treatment.

According to certain aspects, a human or veterinary pharmaceutical composition according to the invention is useful for preventing the bone loss that occurs in the course of aging.

In certain embodiments, a pharmaceutical composition intended for human or veterinary use according to the invention is useful in preventing the bone loss that occurs in the course of aging (osteopenia).

According to certain other aspects, a human or veterinary pharmaceutical composition according to the invention is useful for preventing or treating disorders or pathologies associated with an imbalance of the ratio of bone formation to bone resorption.

In certain embodiments, a pharmaceutical composition intended for human or veterinary use according to the invention is useful in the treatment of physiological disorders connected with an imbalance of bone remodeling, such as osteopenia, type I or type II osteoporosis, secondary osteoporoses, Paget's disease and the bone loss or osteolysis observed near a prosthesis.

The secondary osteoporoses include bone resorption that is caused in physiological situations induced in diseases such as osteosarcoma, rheumatoid arthritis, rheumatoid spondylarthritis, malabsorption syndromes, hypogonadism, primary hyperparathyroidism, diabetes, obesity, chronic obstructive pulmonary disease, chronic liver disease, chronic kidney failure, neurological disorders, AIDS, untreated hyperthyroidism and osteomalacia.

Thus, treatment of secondary osteoporoses relates to the treatment of physiological situations of bone resorption that are not the cause of a disease to be treated, but are concomitant to the disease to be treated. In other words, administration of a phycocyanin composition as defined in the present description to a patient with rheumatoid arthritis does not aim to exert a therapeutic effect on the disease itself. Administration of a phycocyanin composition to this patient aims exclusively to inhibit bone resorption, which is concomitant to the occurrence of rheumatoid arthritis.

It may be a human or veterinary pharmaceutical composition, in particular for dogs or cats, equines such as horses, or else avian species, which includes the farmed avian species such as hens, geese, ducks, turkeys, pigeons etc.

The pharmaceutical composition according to the invention is in a form for oral, parenteral, intramuscular or intravenous administration.

In its form intended for administration in humans, a pharmaceutical composition according to the invention advantageously comprises an amount of phycocyanin suitable for daily administration of the active compound supplied by said composition, varying from 0.01 mg to 10 000 mg.

For administration in humans, a pharmaceutical composition according to the invention comprises an amount of active compound suitable for a daily intake of phycocyanin, supplied by said composition, varying from 0.01 mg to 10 000 mg, preferably from 1 mg to 8000 mg and quite preferably from 10 mg to 6000 mg.

In general, a pharmaceutical composition as defined in the present description is preferably suitable for daily administration of from 0.05 mg/kg to 1000 mg/kg, which includes from 1 mg/kg to 200 mg/kg, for example from 10 mg/kg to 100 mg/kg.

A pharmaceutical composition according to the invention comprises a phycocyanin composition as defined in the present description together with at least one excipient selected from the group consisting of pharmaceutically acceptable excipients.

Techniques for preparing pharmaceutical compositions according to the invention may easily be found by a person skilled in the art, for example in the work Remington's Pharmaceutical Sciences, Mid. Publishing Co, Easton, Pa., USA.

Physiologically acceptable additives, vehicles and excipients are also described in the work titled “Handbook of Pharmaceutical Excipients, Second edition, American Pharmaceutical Association, 1994”.

For formulating a pharmaceutical composition according to the invention, a person skilled in the art may advantageously refer to the latest edition of the European Pharmacopeia or of the United States Pharmacopeia (USP).

A person skilled in the art may notably refer advantageously to the fourth edition 2002 of the European Pharmacopeia, or to the edition USP 25-NF 20 of the U.S. Pharmacopeia (USP).

A pharmaceutical composition as defined above is suitable for oral, parenteral, intramuscular or intravenous administration.

When the pharmaceutical composition according to the invention comprises at least one pharmaceutically or physiologically acceptable excipient, it is in particular an excipient suitable for administration of the composition by the oral route or an excipient suitable for administration of the composition by the parenteral route.

The invention also relates to a method for preventing or treating a disorder associated with an imbalance of bone metabolism, in particular a disorder associated with a loss of bone mass, said method comprising a step in which a therapeutically effective amount of a phycocyanin composition or else of a pharmaceutical composition containing phycocyanin is administered to the patients.

A pharmaceutical composition comprising phycocyanin according to the invention is indiscriminately in a solid form or in a liquid form.

For oral administration, a solid pharmaceutical composition will be preferred, in the form of tablets, soft capsules or hard capsules.

In liquid form, a pharmaceutical composition will be preferred in the form of an aqueous suspension or an oily suspension, or else in the form of a water-in-oil or oil-in-water emulsion.

Solid dosage forms may comprise, as vehicles, additives or excipients, at least one diluent, flavoring, a solubilizer, a lubricant, a suspending agent, a binder, a disintegrant and an encapsulating agent, the identity and the function of these various conventional excipients being fully documented in the European Pharmacopeia or in the United States Pharmacopeia (USP).

In certain embodiments, a solid pharmaceutical composition includes those that are in the form of nanoparticles, which includes nanoparticles of mesoporous silica, functionalized if applicable; which allow controlled release, delayed release if necessary, of the phycocyanin composition.

Such compounds are for example magnesium carbonate, magnesium stearate, talc, lactose, pectin, dextrin, starch, gelatin, cellulosic materials, cocoa butter, etc.

The compositions in liquid form may also comprise water, if necessary mixed with propylene glycol or polyethylene glycol, and optionally also colorants, flavorings, stabilizers and thickeners such as sugars in the liquid forms of the sirup type.

For making a pharmaceutical composition according to the invention, the phycocyanin composition may be prepared according to the teaching of the various patent documents cited above in the description.

Furthermore, the invention is illustrated, but not limited, by the following examples.

### EXAMPLES

### A. Materials and Methods

A.1. Animal study

### A.1.1. Choice of the Animal Model

The experiments were conducted on mice ovariectomized at 8 weeks, a suitable model for studying postmenopausal osteoporosis that has already been used on several occasions for testing nutritional interventions or the potential of vegetable extracts.

### A.1.2. Legal and Technical Aspects

The procedures connected with animal experiments were approved by the Ethics Committee of the Institute, observing the recommendations of the European Union. The mice were housed in the animal house of the Human Nutrition Unit, in a controlled environment: cycle of light and darkness 12 h-12 h, ambient temperature between 20 and 22° C., relative humidity of 50-60%, details of accommodation: one mouse per cage with free access to water.

A standard diet (AIN 93) was supplied by the Unit for Preparation of Experimental Foods (UE0300 UPAE) of the INRA Center of Jouy-en-Josas. Depending on the experimental batches, the mice were supplemented by gavage with two different components:

a) In the first group, supplementation consisted of 500 mg/kg live weight of lysed spirulina (10 mg/d/mouse), supplying 1 mg of phycocyanin.

b) In the second case, the animals received 500 mg/kg live weight of phycocyanin purified from Spirulina (i.e. 10 mg/d/mouse). The dose of spirulina selected was based on the fact that the food supplements available on the market supply up to 4 g of microalgae daily for an average body weight of 60 kg. With regard to extrapolation to the animal, referring to the correspondence of the metabolic weight (P^{0.75}), one mouse will consume 12.5 mg/day of spirulina, or 500 mg/kg of live weight.

Forty-eight female C57BL/6j mice, aged eight weeks, were obtained from the Janvier laboratories (Saint-Berthevin, France). After 3 days of acclimatization, the rodents were distributed randomly into 4 groups of 12 animals:
two groups received a standard diet called “control” (Ctrl),
the other two underwent the test gavages described above (lyophilized Spirulina or Phycocyanin, which was taken up in a volume of 250 μl of normal saline solution, and then administered).

The calculation of power determining the minimum number of animals required for demonstrating a significant effect was established by compilation of data from the literature and studies carried out previously (discriminating characterization of bone mineral density defined as the main criterion).

After 7 days of exposure to these different diets, one of the two groups that received the standard diet underwent sham surgery (SH-Ctrl; placebo group). All the other animals were ovariectomized and assigned to the following groups: OVX-Ctrl, OVX-Spirulina, OVX-Phycocyanin. The surgical interventions were carried out under anesthesia (mixture of Imalgene 1000 (1 g per 10 mL) and Rompun 2% (0.5 g of xylazine per 25 mL)). The mixture injected (using a 26Gx1/2 needle) is 1 mL of Imalgene, 0.5 mL of Rompun, made up to 10 mL with normal saline solution. This 10 mL of solution is calculated for 1 kg of mice, i.e. 0.1 g of ketamine and 0.01 g of xylazine per kg of rodent. One drop of antibiotic is deposited on the wound after the intervention to reduce the risk of infection, then the clamps are fitted. To limit the pain on waking, a subcutaneous injection containing a dose of buprenorphine (buprecar) at 0.1 mg/kg and of metacam at 1 mg/kg, is performed after anesthesia. On waking, the animals had access to water containing acetaminophen (2g/L) for 24 h. Based on an average consumption of 3 mL per day, this acetaminophen solution allows the mouse to ingest 6 mg of acetaminophen in 24 h, or the equivalent of 300 mg/kg of body weight, the LD₅₀ being fixed at 800 mg/kg of body weight in intraperitoneal injection. During this period of 24 h, the prostration and movement of the animal and its food intake were monitored.

The different diets were initiated one week before the surgical intervention. After 6 weeks of experimentation, the rodents were anesthetized. The mixture injected was the same as that used for ovariectomy (1 mL of Imalgene, 0.5 mL of Rompun, made up to 10 mL with normal saline solution). Blood was then taken by intracardiac puncture in a Sarstedt tube, and then left at room temperature for 30 minutes. The tubes were then centrifuged for 5 min at 20° C. and 10000×g. The serum was then collected and divided into aliquots in two different tubes (one for each serum marker to be assayed), which were then stored in a freezer at −80° C. until analysis. After withdrawal of blood, the animal was euthanized directly by cervical dislocation. The liver, spleen and uterus of the animals were taken and weighed, to check for presence or absence of inflammation and to validate ovariectomy. The left and right femurs were also recovered. One femur out of two was placed in formaldehyde solution and stored for one week at 4° C., then transferred to ethanol and finally stored again at 4° C. until densitometer analysis. The other femur was kept directly in tubes in liquid nitrogen, and then frozen at −80° C.

### A.1.3. Analyses Performed

### A.1.3.1. Weighing the Animals and Monitoring Consumption

The weight of the animals was measured once a week. Monitoring of consumption over 2 days was also carried out weekly.

### A.1.3.2. Echo MRI

The operation of Echo-MM is based on the principle of nuclear magnetic resonance imaging, which exploits the magnetic properties of the atoms and makes it possible to determine fat mass, lean mass, free water and total body water of a living, unanesthetized small animal. The rodents go into a transparent plexiglass immobilization tube, the size of which is adapted to the body weight. The mouse goes by itself to the bottom of the tube. A light constraint is then applied using a piston to hold the animal in place at the bottom of the tube. The total immobilization time does not exceed 2 minutes. A single measurement is performed on each individual. Two evaluations of the body composition were carried out with an EchoMRI-900: the first some days before ovariectomy, and the second after 38 days of treatment.

### A.1.3.3. Weight of the Spleen and of the Liver

At sacrifice, the spleen and the liver are carefully removed from the mice and are weighed immediately.

### A.1.3.4. Biomarkers of Bone Metabolism

Bone resorption was evaluated using the ELISA immunoassay kit RatLaps™ EIA (Immunodiagnostic Systems Ltd.) which makes it possible to quantify the C telopeptide of type I collagen (CTX 1), and by means of the ELISA test Ret D Mouse Trance/Rank L/TNFSF11 for Rank L.

With regard to bone formation, the propeptide of type 1 collagen (PINP) was measured using an ELISA kit of the IDS Rat/Mouse PINP EIA type. This specific test makes it possible to determine the release of PINP during synthesis of bone collagen. Osteoprotegerin was assayed using the kit supplied by R&D Systems Quantikine Elisa Mouse OPG/TNFRSF 11b, i.e. an ELISA test. The procedures are supplied by the manufacturers of the kits.

### A.1.3.5. Morphological Analysis of the Bones

Morphological investigation of the femurs was carried out using a micro-CT eXplore CT 120 scanner (GE Healthcare, Little Chalfont, United Kingdom) in the INSERM 990 Mixed Research Unit. Acquisition consists of 360 images of the selected tissue. The left femurs are placed in a PBS buffer solution with a millisecond of exposure per image in an X-ray tube (100 kV and 50 mA). The images are reconstructed using a modified conical beam algorithm with an isotropic voxel of 0.045×0.045×0.045 mm³. The scans obtained were analyzed with the MicroViewH software version 2.3 (General Electric Healthcare Bio-Sciences, Pittsburgh, Pa., USA). A calibration phantom of hydroxyapatite (SB3, Gamex RMI, Wis., USA) serves as a reference for converting the gray scale levels into hydroxyapatite density values. The trabecular bone of the distal portion of the femur was selected for determining bone mineral density and bone volume (BVF=Volume of the bone portion/total volume), after defining a cylindrical region (r=0.7 mm) of interest at the center of the femur, beginning at 0.1 mm from the growth plate and extending to more than 0.32 mm in the proximal direction. The bone mineral density was estimated by the mean value of gray scale levels converted in the region of interest.

### A.1.3.6. Statistical Analyses

The results are expressed as the mean value and the standard error of the mean (SEM). The study was conducted on a total of 13 mice per group (5 groups in total), in order to allow statistical analysis on at least 8 mice per group at the end of the study. The statistical analyses are based on Fisher tests using XLSTAT software (ExcelStat Pro Software, Office Microsoft 2013).

### A.2. In Vitro Study

### A.2.1. Cell Line and Culture Conditions

MC3T3-E1 (immortalized pre-osteoblast line) of mice were cultured in sterile plates at a density of 3×10⁴ cells per cm². The cells were kept in culture medium of the alphaMEM type (GIBCO, Paisley, UK) supplemented with 1% of penicillin/streptomycin (GIBCO, Paisley, UK) and 10% of fetal bovine serum (FBS, Lonza, Levallois-Perret, France). When the cells reached about 80% confluence, they were exposed to the different conditions: either the culture medium alone, regarded as the negative control (C−), or the medium containing 50 g/mL of ascorbic acid and 10 mM of glycerophosphate alone (regarded as the positive control) or else in the presence of phycocyanin at different concentrations (10, 25, 50, 100, 250 μg/ml).

Moreover, RAW 264.7 (immortalized line) of mice (ATCC, Washington, D.C., USA) were used as models of pre-osteoclasts and were induced with Rank L. The cells were seeded in the wells at a density of 1×10⁴ cells/m² and were kept in culture medium (α-minimal essential medium α-MEM; GIBCO, Paisley, UK) supplemented with 1% of penicillin/streptomycin (GIBCO, Paisley, UK) and 10% of fetal bovine serum (FBS). When the cells reached about 80% confluence, they were exposed to different conditions: either the culture medium alone, regarded as the negative control (C−), or the culture medium containing either a culture medium alone, regarded as the negative control (C−), or culture medium containing 50 ng/mL of receptor activator of nuclear factor-kappa B ligand (RANK-L) (R&D Systems) alone (regarded as the positive control), or else in the presence of phycocyanin at different concentrations (5, 10, 25, 50, 100 μg/ml).

The two cellular types were cultured at 37° C. in a humid atmosphere at 5% CO₂ in the air. The medium was changed every 2 days.

A. 2.2. Cell proliferation

Measurement of cell proliferation is based on a colorimetric assay. An XTT yellow tetrazolium salt is reduced by the succinate dehydrogenase activity of the viable cells, in the presence of an electron coupling reagent. The reaction produces an orange-colored formazan salt.

The RAW264.7 and the MC3T3-E1 were seeded in 96-well plates at a density of 3.5×10³ cells per well, and were then cultured for 2 hours with:

a) in the case of MC3T3-E1, 10% of serum alone, 2% of serum alone or with phycocyanin at a concentration of 100, 250, 1000, 2500 μg/ml;

b) in the case of RAW 264.7, 10% of serum alone, 2% of serum alone or with phycocyanin at a concentration of 10, 25, 50, 100, 250, 1000 μg/ml.

Cellular viability was determined at 24 hours by a method based on XTT using a cell proliferation kit (Cell Proliferation Kit II Sigma-Aldrich, St. Louis, Mo., USA), following the manufacturer's recommendations. OD was determined at 450 nm.

### A. 2.3. Measurement of the Activity of Alkaline Phosphatase (ALP)

Alkaline phosphatase is an early marker of bone formation. Measurement of its activity is based on the capacity of the enzyme to catalyze the hydrolysis of p-nitrophenylphosphate (p-NPP) to p-nitrophenol, a chromogen with absorbance at 405 nm.

The enzymatic activity of ALP was measured on the osteoblasts at 0, 2, 7 and 14 days according to the method published previously, adapted to our experimental conditions. The osteoblast culture is rinsed twice with PBS (Sigma-Aldrich, 38297 Saint-Quentin Fallavier, France), and then kept at −20° C. Then the cells are lysed and homogenized in diethanolamine/magnesium chloride hexahydrate buffer (pH 9.8; Sigma-Aldrich). The cellular lysate (10 μl) is added to 200 μl of p-nitrophenyl phosphate solution (6 mg/ml) (Sigma-Aldrich, St. Louis, Mo., USA). The absorbance is measured at 405 nm in kinetic conditions (every 150 s for 30 min), at 30° C., using an ELX808 microplate reader (BioTek Instruments Inc, Winooski, Vt., USA). Finally the proteins are measured using the BioRad protein assay (BioRad, Munich, Germany).

ALP is expressed in micromoles of p-nitrophenol per hour and per milligram of proteins.

### A. 2.4. Measurement of the Activity of Tartrate-Resistant Acid Phosphatase (TRAP)

Tartrate-resistant acid phosphatase is described as an enzyme marker of osteoclasts, which resorb bone tissue. This enzyme, also called type 5 acid phosphatase, exists as the isoforms 5α and 5β, the latter exclusively being recognized as the 5β active isoform of TRAP specific for osteoclasts. The inactive part of the 5β fraction is produced by the macrophages.

The activity of TRAP is measured in accordance with the standard method using a leukocyte acid phosphatase kit (Sigma Aldrich).

After culture for 3 days, the cells lysed with NP 40 lysis buffer are incubated at 37° C. in a p-nitrophenyl phosphate buffer, with 125 mM of sodium acetate buffer (pH 5.2), and 100 mM of p-nitrophenyl phosphate.

The absorbance is measured at 37° C. at 405 nm in kinetic conditions at 37° C. using a plate reader.

For each sample, the concentration of protein is measured and TRAP is expressed in OD/min/mg protein.

A. 2.5. TLDA and Method of Obtaining the cDNAs

### A.2.5.1. Extraction of the RNAs

The extraction, the aim of which is to recover the elements necessary for analyzing the expression of the transcripts, takes place in several steps. The ribonucleic acids (RNAs) are purified following extraction with phenol-chloroform (Trizol-Roche), and then assayed.

### A.2.5.2. Retro-Transcription of the RNAs

Strictly aseptic conditions are required to avoid the application of false positives and contamination. Reverse transcription was carried out using High capacity cDNA reverse transcription Kits (applied). For each sample, 20 μL of solution was prepared and was deposited in ice. The first step consists of preparing 2× reverse transcription master mix, in ice, consisting of DNA polymerase (enzyme performing retro-transcription of an RNA to cDNA), a buffer for optimizing enzymatic activity, primers for fixing polymerase on the RNA strand and of course nucleotides.

In a second step, 10 μL of mix is added to 10 μL of a mixture of water and RNAs extracted in the preceding step (the proportions in the mixture being adjusted so that the same concentration of RNA is present in each of the conditions at a level of 500 ng), in the well of a 96-well plate.

After centrifugation of the plate, the sample is placed in the TC 512 TECHNE thermocycler for initiating the reaction with a program adapted for performing TLDA. 10 minutes at 25° C., then 120 minutes at 37° C. and finally 5 minutes at 85° C.

### A.2.5.3. TLDA

The aim is to amplify and quantify the target sequence. The TLDA technology (or TaqMan® Array) makes it possible to quickly determine the expression profile of a large number of genes or of miRNA, on several samples, in a small reaction volume. Its high sensitivity and its reproducibility make it a powerful tool for performing analyses from very small amounts of starting material, and it is therefore suitable for microgenomic studies. The TLDA® is a microfluidic card comprising 384 wells. It makes it possible to analyze simultaneously from 1 to 8 samples on 11 to 380 different primer systems per run of real-time PCR (2.5 h).

In this study, we used TLDA cards specially designed for exploration of 48 genes implicated in the process of bone resorption in mice. For each sample, we prepared a solution of 100 μl kept in ice with 50 μl of TaqMan® Fast advanced Master Mix and 50 μl of cDNA in water. Before they are put in the apparatus (Applied Biosystems 7900 HT real time PCR system) and before proceeding with the analysis using the SDS software, the cards are centrifuged 2 times for 1 minute at 1200 rpm.

### A.2.5.4. Statistical Analyses

The results are expressed as mean values±their standard errors (SEM).

All the data were analyzed using the XLSTAT software (Addinsoft, Paris, France).

Statistical analysis of the ANOVA type (parametric test) revealed the differences between groups. If the result was judged to be significant (P<0·05), the multiple comparison Fisher test was then used to determine the specific differences between the mean values.

### Example 1: In-Vivo Effect of Phycocyanin on Bone Resorption

### 1.1. Study Validation

The significant decrease in weight of the uterine horns in the OVX control group relative to SH-Ctrl allows validation of the quality of castration. In the ovariectomized mice, this parameter was not altered significantly by the different experimental regimes.

### 1.2. Weighing the Animals and Monitoring Consumption

The daily weight gain (DWG) was evaluated daily for the first 7 days of treatment, and then weekly. Starting from the third week, this parameter is significantly higher in the OVX-Ctrl relative to the SH-Ctrl.

The results obtained show that the different diets (or gavage) did not cause a significant change of this criterion.

Monitoring of consumption, carried out weekly throughout the experiments, does not show any significant difference between the two groups on the standard diet (SH-Ctrl and OVX-Ctrl). Castration does not alter food intake. The same applies to the ovariectomized animals, receiving the standard diet, and despite gavage.

The results obtained did not show that the administration of phycocyanin caused a change in the weight of the animals or in their food consumption.

### 1.3. Echo-MRI

At the end of the experiments, fat mass was shown to be significantly higher in the OVX-Ctrl, relative to the SH-Ctrl. In contrast, consumption of the different diets did not have a statistically significant effect in the ovariectomized animals.

Thus, the results did not show an effect from administration of phycocyanin on the animals' fat mass.

### 1.4. Weight of the Spleen and Liver

At sacrifice, in the OVX-Ctrl group this parameter is statistically higher than is observed in the SH-Ctrl group. Inflammation following the surgical intervention could therefore be detected. However, it was not normalized by the experimental interventions since no significant difference is observed within the different OVX groups.

The different experimental conditions, with or without administration of phycocyanin, did not significantly alter the average weight of the livers.

### 1.5. Biomarkers of Bone Metabolism

With regard to OPG, which constitutes a marker indicative of inhibition of bone resorption, significantly higher values are found in the SH-Ctrl group relative to the OVX-Ctrl group, as well as in the phycocyanin OVX group relative to the OVX-Ctrl group, compared to the OVX-Ctrl group.

Consequently, the results show that phycocyanin at least partially normalizes the increase of the marker OPG that is induced by the surgical intervention.

In contrast, the marker RankL, which constitutes a marker indicative of bone resorption, is increased by castration. Administration of phycocyanin did not provide normalization of the level of this marker.

The OPG/RANKL ratio, which constitutes an index of bone remodeling, is significantly higher in the SH-Ctrl group relative to the measured value of this ratio in the OVX-Ctrl group.

The OPG/RANKL ratio is increased in the ovariectomized rodents to which phycocyanin was administered, which makes it possible to orient metabolism favorably for preservation of the bone pool.

With regard to the marker PINP, which constitutes a marker indicative of bone formation, no statistically significant difference is observed between the different experimental groups.

The same results are observed with respect to the marker CTX 1, which is a marker indicative of bone resorption.

### 1.6. Analysis of the Bone Mass

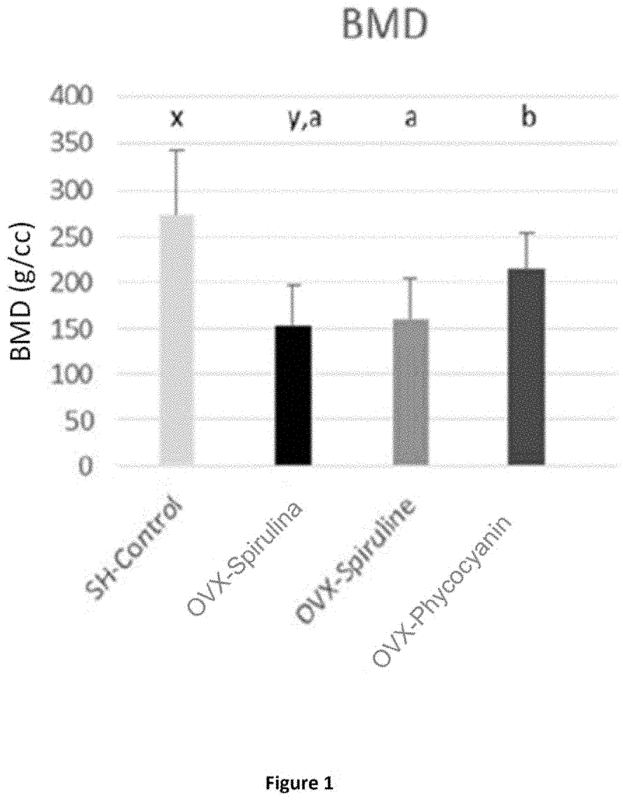

The bone mass was measured in the different groups of animals. The results are presented in FIG. 1.

Analysis of the bone mineral density (BMD) confirms the decrease, observed conventionally, of this parameter in the OVX-Ctrl mice relative to the SH-Ctrl mice.

Moreover, only the group OVX that was administered phycocyanin has a bone mineral density significantly higher than that of the OVX control batch.

These results show that the administration of phycocyanin makes it possible to block bone resorption in the animals in which a hormone deficiency was caused experimentally by ovariectomy.

### Example 2: In-Vitro Confirmation of the Effects of Phycocyanin on Bone Resorption

The various experiments carried out in vitro were conducted with increasing doses of phycocyanin.

### 2.1. Cell Proliferation

The proliferation of the osteoblasts was measured by XTT after 24 h of induction in the presence of different concentrations of phycocyanin.

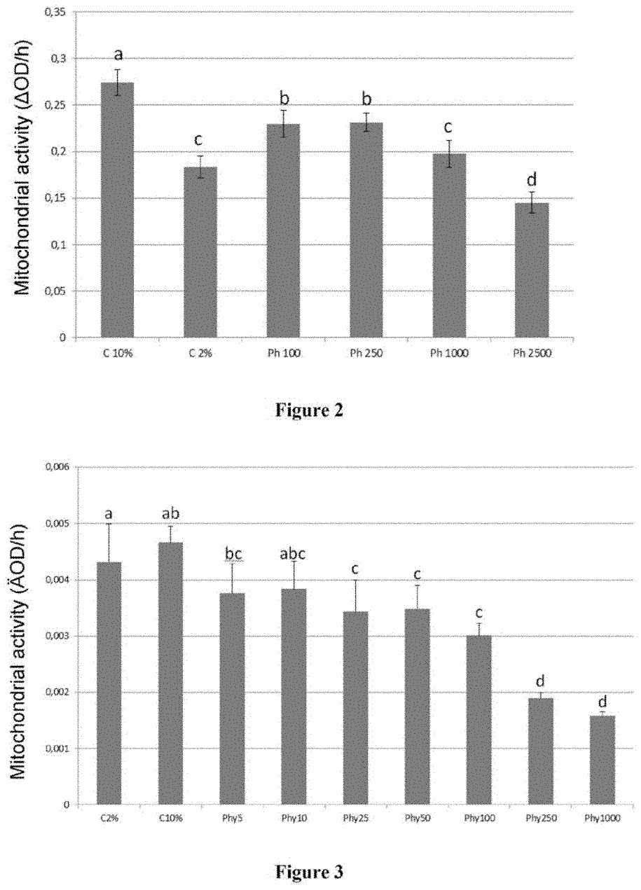

The greatest effect was observed in the cells exposed to doses of 100 μg/ml and 250 μg/ml of phycocyanin, relative to the control group 2%. (FIG. 2)

In contrast, a concentration of 2500 μg/ml proved inhibitory, whereas a dose of 1000 μg/ml did not have a statistically significant effect. (FIG. 2)

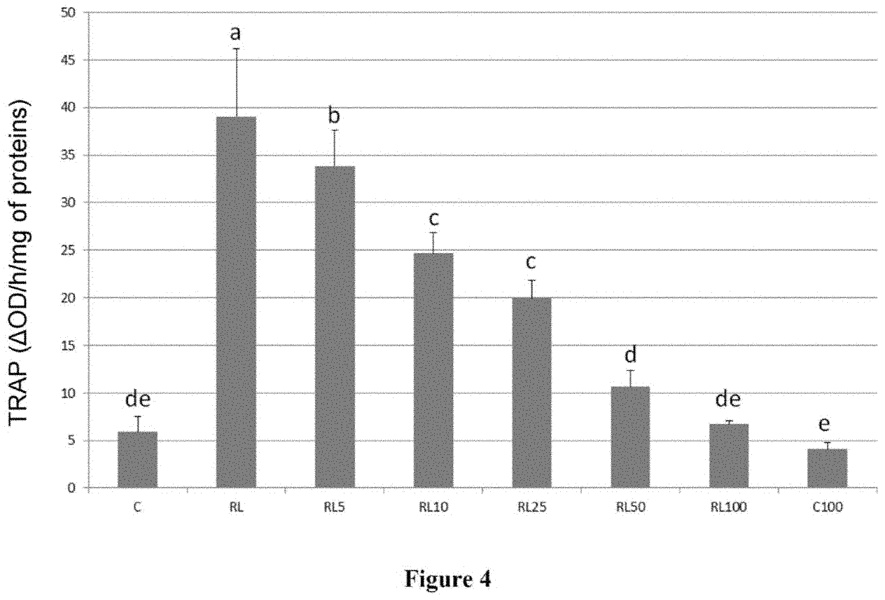

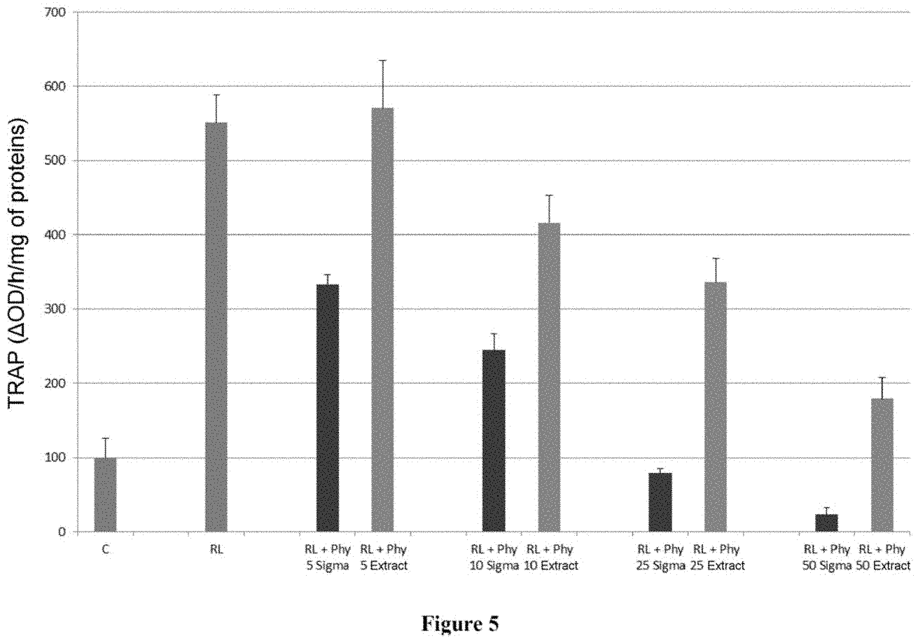

With regard to the osteoclasts, there was dose-dependent inhibition of proliferation in the cell cultures exposed to phycocyanin. (FIGS. 3 and 4)

These results show that phycocyanin has an inhibitory effect on proliferation of osteoclasts, which is in agreement with the in-vivo results, which show that phycocyanin has an inhibitory effect on bone resorption.

### 2.2. Measurement of the Activity of Tartrate-Resistant Acid Phosphatase (TRAP)

The activity of tartrate-resistant acid phosphatase (TRAP) was measured in cultures of osteoclasts. The results are presented in FIG. 4.

The results in FIG. 4 show that in the course of differentiation of the osteoclasts, the measurement of enzymatic activity of TRAP is significantly higher in the RankL control group relative to the control. This phenomenon is inhibited dose-dependently by phycocyanin.

### 2.3. TLDA

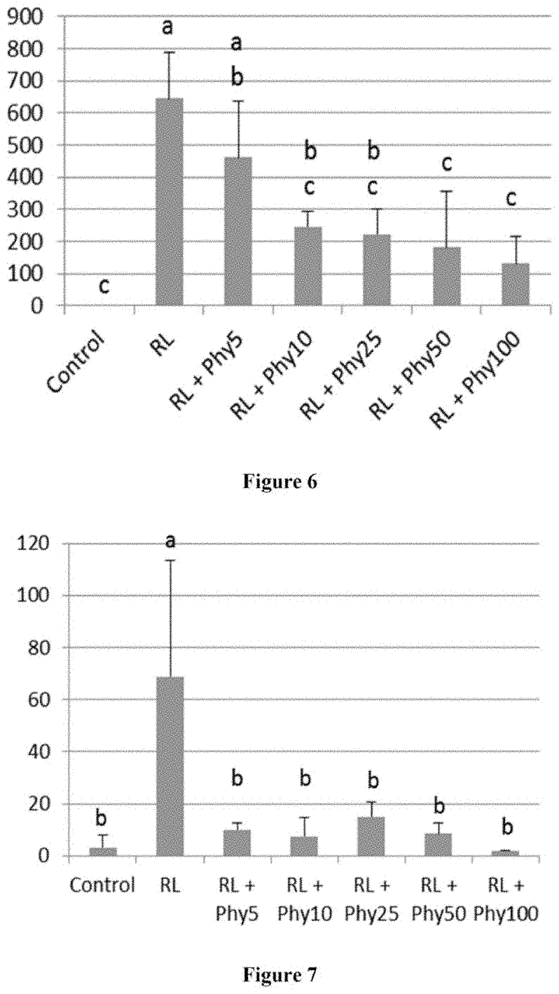

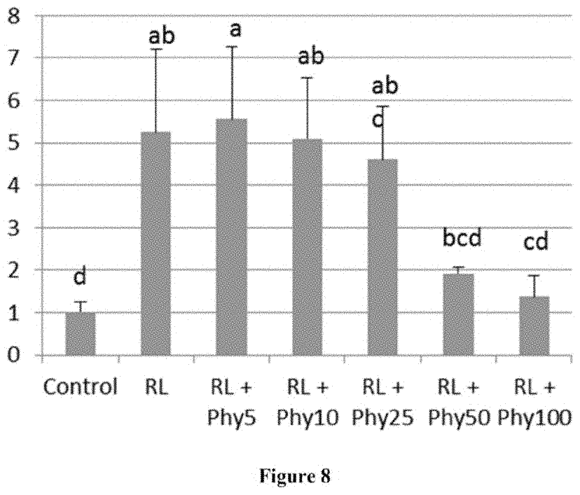

To study the effect of phycocyanin on bone resorption, we measured the expression of various markers, respectively (i) the marker TRAP indicative of cellular differentiation (FIG. 6), (ii) the marker Nox4 indicative of oxidative stress (FIG. 7) and (iii) the marker NFATC1, which is a transcription factor (FIG. 8).

TLDA analysis was carried out on mRNA extracts of the cultures of the Raw 264.7 line, which is a model cell line of pre-osteoclasts. The cultures of cells of the Raw 264.7 line were exposed to phycocyanin.

The results show:
a decrease in the level of the specific markers of the osteoclasts (such as TRAP (FIG. 6), the calcitonin receptor, the metalloproteases),
oxidative stress, with a decrease in the expression of Nox 4 (see FIG. 7),
a decrease in expression of the transcription factor NFATC1, in the presence of a phycocyanin concentration of 100 μg/ml (see FIG. 8).

### Summary of the Experimental Results

The animal experiments conducted on ovariectomized mice, aged 8 weeks, revealed a potentially beneficial action on the bone, of daily supplementation with phycocyanin, at a dose of 500 mg/kg body weight. In fact, the results of measurement of the femoral trabecular BMD indicate that this nutritional intervention makes it possible to prevent, partly, the bone loss induced by the reduction in estrogen impregnation, as well as the biological markers of bone remodeling such as OPG.

To validate these results in vivo, experiments were also conducted on immortalized murine lines of osteoblasts and of osteoclasts exposed to various concentrations of phycocyanin.

This compound essentially targets the differentiation and proliferation of the osteoclasts with a dose-dependent decrease of synthesis of TRAP and a dose-dependent decrease of expression of TRAP, MMP9, the calcitonin receptor, and of NOS 2 and NFATc1. To a smaller extent, there is a pro-proliferative action on the osteoblasts.

1. A method for inhibiting bone resorption in humans or animals comprising administering a phycocyanin composition to said humans or animals.

2. The method according to claim 1, wherein the said phycocyanin composition is comprised in a nutritional composition suitable for oral administration.

3. The method according to claim 2, wherein the said composition is intended to prevent the bone loss that occurs with aging.

4. The method according to claim 2, wherein the said composition is intended to prevent or treat disorders selected from type I or type II osteoporosis, secondary osteoporoses, Paget's disease, and the osteolysis observed near a prosthesis.

5. The method according to claim 2, wherein the said nutritional composition is in the form of food compositions, drinks, including juices (of fruits or vegetables or algae), vegetable milks, oils, butters, margarines, vegetable fats, canned food, soups, milk-based preparations, ice creams, cheeses, desserts, confectionery products, cereal bars, breakfast cereals, condiments, products for seasoning foodstufs.

6. The method according to claim 2, wherein the said nutritional composition is in the form of a product intended as animal feed, in wet, semi-wet or dry form.

7. The method according to claim 1, wherein the said phycocyanin composition is in the form of an extraction product obtained from spirulina.

8. The method according to claim 2, wherein the said nutritional composition is suitable for daily administration of from 0.05 mg/kg to 1000 mg/kg.

9. The method according to claim 1, wherein the said phycocyanin composition is comprised in a human or veterinary pharmaceutical composition.

10. The method according to claim 9, wherein the said pharmaceutical composition is intended to prevent the bone loss that occurs in the course of aging.

11. The method according to claim 9, wherein the said pharmaceutical composition is intended for preventing or treating a pathology associated with an imbalance of the ratio of bone formation to bone resorption.

12. The method according to claim 9, wherein the said pharmaceutical composition is intended for preventing or treating a pathology selected from type I or type II osteoporosis, secondary osteoporoses, Paget's disease, bone loss or osteolysis observed near a prosthesis.

13. The method according to claim 9, wherein the said pharmaceutical composition is in a form for oral, parenteral, intramuscular or intravenous administration.

14. The method according to claim 9, wherein the said pharmaceutical composition is suitable for daily administration of from 0.05 mg/kg to 1000 mg/kg.
